(19) United States
(12) Patent Application Publication
(10) Pub. No.: US 2004/0198643 A1
(43) Pub. Date: Oct. 7, 2004

(54) Antibacterial agent

(76) Inventor: Frank Mayer, Bovenden (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/476,385

(22) PCT Filed: Apr. 22, 2002
(86) PCT No.: PCT/EP02/04410

(30) Foreign Application Priority Data
Apr. 30, 2001 (DE) 101 21 145.7

(51) Int. Cl. A61K 38/17; A61K 38/12

(52) U.S. Cl. 514/9; 514/12

(57) ABSTRACT

The invention concerns the use of substances that bind to the bacterial translation factor EF-Tu to inhibit the formation of a cytoskeleton in bacterial cells and for the production of antibacterial agents. In addition the invention concerns antibacterial agents which contain partial sections of the amino acid sequences of domains 2 and/or 3 of a bacterial EF-Tu protein having a length of preferably 4-20 amino acids.

The invention concerns the use of substances which bind to the bacterial translation factor EF-Tu for inhibiting the formation of a cytoskeleton in bacterial cells and for producing antibacterial agents. The invention also concerns antibacterial agents which contain partial sections of the amino acid sequences of the domains 2 or/and 3 of a bacterial EF-Tu protein preferably having a length of 4-20 amino acids.

Penicillin or other antibiotics which have a specific inhibitory effect on growing bacterial cells have, among others, previously been used as antibacterial agents. This effect is based on an inhibition by these antibiotics of the extension of the peptidoglycan skeleton that is necessary for cell growth. Growing cells are considerably weakened by this destabilization of the murein. Bacteria in the stationary phase are not inhibited because the murein skeleton is not extended in this phase.

The bacterial protein EF-Tu contains the domains 1, 2 and 3 (Song, H., Parsons, M. R., Rowell, S., Leonard, G., Phillips, E. V., J. Mol. Biol. 285, 1245-1256, 1999). The sequences of the protein EF-Tu and its encoding gene have been published for Escherichia coli and a number of other eubacteria and are accessible in databases. It has also been described that the domain 1 of EF-Tu plays a role in protein synthesis.

The possible existence of a permanent prokaryotic cytoskeleton has been discussed in Naturwissensch. 85, 1998, 278-282 (Mayer et al.). However, an involvement of the bacterial protein EF-Tu in the formation of such a cytoskeleton was unknown.

With regard to the location of EF-Tu in bacterial cells, it has previously been assumed in the literature (cf. e.g. Schilstra, M. J., Slot., J. W. van der Meide, P. H., Posthuma, G., Cremers, A. F., Bosch, L.: Immunocytochemical localization of the elongation factor Tu in Escherichia coli cells., Biochem. Biophys. Acta 1291, (1996), 122-130) that EF-Tu is distributed almost homogeneously in the cytoplasm. However, previous experiments did not take into account the fact that artificially produced EF-Tu fibrils can be depolymerized in vitro by low temperatures.

It was surprisingly found that a cytoskeleton exists in prokaryotic cells which can be stained with anti-EF-Tu antibodies. This cytoskeleton comprises a network of protein fibrils which are located near to the surface of the cytoplasmic membrane that faces the cytoplasm and they extend through the cytoplasm. The cytoplasmic membrane and the peripheral part of this network can be regarded as two concentric hollow tubes where the cytoplasmic membrane represents the outer of the two tubes and the peripheral part of the network (cytoskeleton) represents the inner tube. Fibrils running through the cytoplasm complement and stabilize the system and are attachment sites for ribosomes. Ribosomes have also been detected in the peripheral part of the cytoskeleton oriented towards the cytoplasm.

Hence the prokaroytic cytoskeleton has several variants:

Variants which mediate special functions consisting of proteins that are similar to the actin of higher cells and which, in the case of rod-shaped bacteria, define the length and diameter of the cell,

variants which consist of proteins that are similar to the tubulin of higher cells and ensure controlled cell division and

a variant which occurs generally in all prokaryotes (basic cytoskeleton) which consists of a network of protofilaments of the protein EF-Tu (elongation factor Tu) which the cell uses as form-stabilizing structural elements and which act as an attachment structure for ribosomes and other complex molecular aggregates. The last variant is also referred to herein as a cytoskeletal network.

EF-Tu is a protein which contains three domains of which domain 1 is involved in the translation process. Up to now no specific function has been described for domains 2 and 3. It has now been found that the laterally exposed epitopes of domains 2 and 3 form a fit in which one surface is convex and one surface is concave. It is assumed that these fits can result in the formation of EF-Tu polymers and especially a linear arrangement of fibrils in vitro as well as in vivo. These fibrils are the components of the network which act as a cytoskeleton. Hence substances which bind to EF-Tu especially in the region of domains 2 or/and 3 could be used to inhibit the formation of a cytoskeleton in bacterial cells and thus to produce an antibacterial agent.

Hence the cytoskeletal network could thus be used as a target for a new class of antibiotics.

In particular EF-Tu can be used as a target protein for new bacterial agents which can occupy the fitting sites of domains 2 or/and 3 and thus prevent the formation of EF-Tu polymers in the cell which are essential for the structure of the bacterial cell.

This mode of action is fundamentally different from the mode of action of other antibiotics acting on EF-Tu (cf. e.g. Vogeley, L., Palm, G. J., Mesters, J. R., Hilgenfeld, R.: Conformational change of elongation factor Tu (EF-Tu) induced by antibiotic binding. J. Biol. Chem. 276 (2001), 17149-17155). This publication shows that the action of previously known antibiotics of the kirromycin type is due to the fact that they prevent the reversibility of a conformational change of domain 1 resulting in a bending of domain 1 towards domain 2 when GTP is bound. This mechanism is fundamentally different from the mechanism of action described herein of an inhibition of polymerization in which domains 2 and 3 are involved.

EF-Tu comprises 394 amino acids. Amino acids 8-204 belong to domain 1 and amino acids 172-204 form a linking structure to domain 2. Amino acids 205-298 belong to domain 2 and domain 3 comprises the amino acids 299-394.

Different secondary structures occur within domains 2 and 3. In this context the amino acid sequences of 317 to 328 and of 343 to 354 which are located in domain 3 are of particular interest since they form loops which protrude freely into space and are candidate sequences for an interaction with amino acid sequences which are located in a depression on a corresponding position on the periphery of domain 2 where these sequences extend from amino acid 218 to 224.

It was surprisingly found according to the invention that in the case of the bacterial cytoskeleton it is basically possible to damage the cells by inhibiting the polymerization of EF-Tu. In particular such cell damage is also achieved in common bacterial cells which have a cell wall. The invention is particularly applicable to eubacteria.

A wide variety of substances can be used to inhibit the formation of cytoskeletons provided they are able to inhibit the interaction between domain 2 and domain 3 of two neighbouring EF-Tu molecules. Suitable substances can for example be identified by a method comprising:

(a) contacting a substance to be tested with bacterial EF-Tu or with a partial fragment thereof capable of polymerization such as a fragment which contains the domains 2 and 3 and

(b) determining whether the substance can inhibit the formation of EF-Tu polymers.

This method can be carried out in vitro as well as in vivo. In an in vitro method purified EF-Tu molecules or suitable partial fragments thereof are preferably incubated under conditions in which fibril formation can take place. The effect of a test substance on fibril formation can be determined in a simple manner for example by means of immunological staining using labelled anti-EF-Tu antibodies or by using EF-Tu molecules which carry a marker group e.g. a fluorescent marker group. Of course the method can also be carried out in vivo in which case the effect of adding a test substance on the fibril network in a cell can be determined by immunological methods e.g. immunohistochemically using labelled anti-EF-Tu antibodies and microscopic evaluation.

Substances which inhibit the formation of EF-Tu polymers and which can be obtained by the method described above as well as substances derived therefrom e.g. by means of empirical derivatization or/and by computer modelling, can be formulated as a pharmaceutical composition optionally together with common pharmaceutical vehicles, auxiliary substances or/and diluents.

The pharmaceutical composition can for example be present as a liquid preparation, solid preparation, emulsion or dispersion. Depending on the preparation it can be administered by injection or orally, rectally, nasally, topically etc. The dosage is selected depending on the active substance, the form of administration and the type and severity of the disease, such that it is possible to combat bacterial infections.

The antibacterial agent can have a variety of effects. On the one hand substances are used which can bind directly to the fitting sites of domains 2 or/and 3 of EF-Tu. On the other hand it is also possible to use substances which bind to other positions on the EF-Tu molecule but have an inhibitory effect on the fitting and thus preventing fibril formation.

Peptidic, antibacterial agents are used in a preferred embodiment of the invention. The peptidic agents are based on oligopeptides which bind to EF-Tu preferably in the region of the sites of fit of domains 2 or/and 3. These oligopeptides may contain partial sections of the amino acid sequences of domains 2 or/and 3 having a length of preferably 4 to 20 amino acids, particularly preferably 5 to 15 amino acids and especially preferably having a length of 6-12 amino acids. These partial sections are able to bind to complementary sequences of the other domain i.e. sequences from domain 2 are able to bind to domain 3 and sequences from domain 3 are able to bind to domain 2.

In another preferred embodiment the substances which bind to EF-Tu contain a partial section of the amino acid sequences from domain 2 having a length of at least 4 and in particular of at least 5 amino acids, in particular partial sections in the region of domain 2 of amino acids 218 to 224 and at the same time no section which corresponds to the region of amino acids 317 to 328 or/and the region of amino acids 343 to 354 of domain 3 of EF-Tu. Alternatively substances are preferred which contain partial sections of the amino acid sequences from domain 3 having a length of at least 4 amino acids, in particular of at least 5 amino acids and particularly preferably of at least 6 amino acids and no partial sections corresponding to amino acids 218 to 224 of domain 2. Such sections can for example be a truncated EF-Tu which is composed only of domain 3 without domains 1 and 2 or only of domains 1 and 2 without domain 3. Such an EF-Tu fragment competes in the cell with the natural EF-Tu protein molecules synthesized by the cell and results in chain termination when it is incorporated into the polymerizing protofilament since in each case the second domain required for chain extension is absent. As a result an intact network is no longer formed. This is synonymous with the loss of viability of the bacterial cell. A disorder in the development of the network in the bacterial cell has an adverse effect on the shape and behaviour of the bacterial cell as demonstrated by experiments. The adverse effect on the shape and behaviour or the cell indicates the expected cell death which occurs when the antibiotic according to the invention is used.

Instead of the described truncated EF-Tu fragment, it is also possible to use an antibiotic which prevents the polymerization of EF-Tu protein molecules i.e. a chain termination, by other means for example due to the presence of sections which prevent binding of further EF-Tu protein molecules.

A particular advantage of the antibiotics according to the invention is that there is only a slight risk of the development of bacterial resistance to this new class of antibiotics. A resistance would mean that the bacterium would degrade the peptide that has been transferred to the inside of the cell. If this were to occur, the bacterium would not be able to avoid also degrading its own structurally identical peptide which is a component of the cell's EF-Tu protein and which is of major importance for translation.

The antibacterial agents can comprise linear or cyclic peptidic compounds or peptidomimetics. Peptidic compounds can be composed of natural L-α-amino acids, but also other amino acids e.g. D-α-amino acids, azamino acids, β-amino acids, non-genetically coded L- or/and D-α-amino acids etc. or combinations thereof. The preparation of peptidomimetics is described for example in RIPKA, A. S., RICH, D. H. (1998) Peptidomimetic design, Curr. Op. Chem. Biol. 2, 441-452.

In addition the peptidic compounds or peptidomimetics may contain bound hydrophobic groups which facilitate transfer through the cytoplasmic membrane or very bulky groups which prevent the attachment of further EF-Tu molecules and thus prevent the formation of a polymerization product. The antibacterial agents may also carry groups which protect against degradation.

The antibacterial agents can be used against any prokaryotic organisms and archaea and especially pathogenic organisms. Gram-positive bacteria, Gram-negative bacteria and mycoplasma have a cytoskeleton based on EF-Tu and can therefore be combated by the agent according to the invention. For example antibacterial agents against vancomycin-resistant microorganisms e.g. staphylococci can be used.

Hence the new class of antibiotics has a wide spectrum of applications. It was found that the regions which are responsible for binding the monomers to form the protofilaments, have a very similar amino acid sequence in all examined bacteria. EF-Tu is highly conserved in this region. Also the distances between these regions in a given EF-Tu molecule i.e. the distances between the exposed regions of domains 2 and 3 are identical in terms of the number of amino acids and there are always 126 amino acids between the conserved regions.

The antibiotics according to the invention are characterized by a high specificity and especially by very low side-effects. Large EF-Tu sequences do not occur in the human cell apart from in mitochondria. The mitochondrial EF-Tu-like sequences are substantially protected from the antibiotic by the double membrane of the mitochondria.

The invention is illustrated by the following figures and examples.

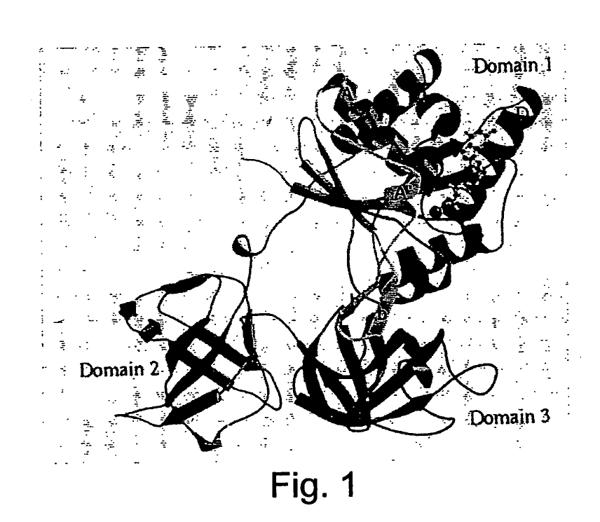

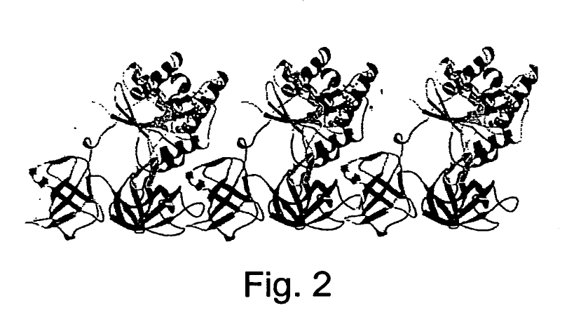

FIG. 1 shows the macromolecular architecture of the bacterial protein EF-Tu in which domains 1, 2 and 3 are described in more detail. This bacterial protein EF-Tu can associate during polymerization to form periodically structured fibrils as shown in FIG. 2.

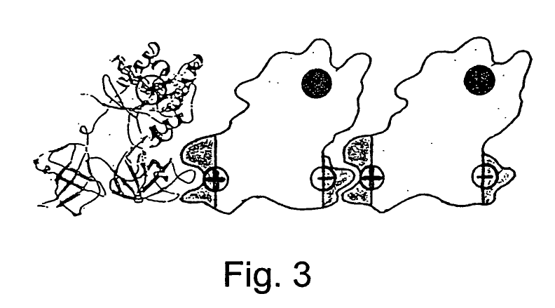

FIG. 3 shows a schematic representation of the polymerization at the reactive binding regions of domains 2 and 3 labelled with + or −.

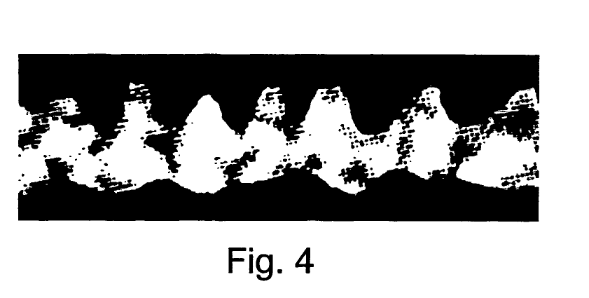

FIG. 4 shows an enlargement (enlargement: ca. 1.5 million) of an electronmicrograph of an in vivo polymerized isolated fibril from EF-Tu protein molecules. The domains 1 are located above the dotted line and the juxtaposed domains 2 and 3 are below.

If the polymerization of the EF-Tu protein is repressed at the binding regions labelled with + and − in FIG. 3 by adding an excess of particles containing partial sections of the amino acid sequence of domains 2 or 3, the affected bacterial cell is unable to survive because the cell structure breaks down.

### EXAMPLE

Experiments were carried out on the Gram-positive bacterium Thermoanaerobacterium thermosaccharolyticum EM1 (abbreviated EM1 in the following) and the bacterium Mycoplasma pneumoniae (abbreviated Mp in the following) which lacks a cell wall proving that these bacteria have a permanent cytoskeleton that is based on EF-Tu.

The experiments included the identification and cellular localization of candidate proteins for such a bacterial cytoskeleton by using anti-actin antibodies (prepared against actin of higher cells) which cross-react to a greater or lesser extent with bacterial proteins due to the fact that bacteria are known to have proteins which belong to the actin super family without having conspicuous sequence homologies with actin of higher cells. Prokaryotes do not have distinct actin genes.

In addition to immunoelectron microscopy with the aforementioned antibodies on ultrathin sections through bacteria, whole mount techniques were also used. It was found by a combination of these techniques that a network of protein fibrils is located near to that surface of the cytoplasmic membrane which faces the cytoplasm and extend through the cytoplasm. The components of these fibrils cross-react with the anti-actin antibodies. The cytoplasmic membrane and the peripheral part of this network form two concentric hollow tubes where the cytoplasmic membrane forms the outer of the two tubes and the peripheral part of the network (cytoskeleton) forms the inner part. The fibrils extending through the cytoplasm complement and stabilize the system and are attachment sites for ribosomes. Ribosomes also sit on the peripheral part of the cytoskeleton oriented towards the cytoplasm.

The cells of the EM1 cells were disrupted with the aid of a French press and the material obtained (soluble fraction, particulate fraction) was subjected to SDS gel electrophoresis and Western blotting. Several defined bands were obtained in the SDS gel of which one band (at about 43 kDa) could be stained with anti-actin antibodies as well as with anti-EF-Tu antibodies obtained against the EF-Tu of Mp. This band was particularly pronounced where the particulate fraction of cell lysis obtained by low-speed centrifugation had been used as the material for SDS gel electrophoresis. Anti-EF-Tu antibodies were used because EF-Tu is usually found at 43 kDa (comprises about 9% of the protein mass of a bacterium), because EF-Tu belongs to the actin superfamily and because EF-Tu occurs in large amounts in a prokaryotic cell.

The role of EF-Tu as a structural component of a bacterial cytoskeleton is new. This property of EF-Tu as a structural component of a complex network like that of the cytoskeleton infers that the bacterial cell has to use a large amount of protein for this purpose. A comparison of the structure of this bacterial cytoskeleton with that of higher cells makes it clear that the bacterial cytoskeleton must also be composed of several types of protein. EF-Tu is a major component. Although in higher cells EF-Tu is not involved in the formation of the cytoskeleton (the higher cell has no EF-Tu), it is, however, known that a large number of different proteins contribute to the formation of the cytoskeleton.

In the section and in the whole mount it was possible to show that components of the above-mentioned network of the cytoskeleton that react with anti-actin antibodies also intensively reacted with anti-EF-Tu antibodies.

This reaction occurred in the case of Mp with the entire originally covered surface that was exposed to the environment by Triton treatment (removal of the cytoplasmic membrane). A control that was prepared using cells which were not treated with Triton but otherwise treated identically and thus had not lost their cytoplasmic membrane, exhibited no labelling. Hence in this control experiment the cytoplasmic membrane masked the potential binding sites for EF-Tu.

The surface exposed by removing the cytoplasmic membrane is the peripheral part of the cytoskeleton of the cell. This, however, does not expose inner cell components such as ribosomes which have been shown to be the attachment sites for EF-Tu which perform a helper function during translation (domain 1 of EF-Tu acts in this case). As a result it was concluded that during the course of translation EF-Tu does not go to the ribosome but the ribosome goes to EF-Tu which, due to its property as a component of the cytoskeleton, is spatially fixed on the cell periphery and on fibrils that run crosswise through the cytoplasm.

The occurrence of a permanent bacterial cytoskeleton was also detected in the eubacteria Escherichia coli, Bacillus sp., Ralstonia eutropha and Thermoanaerobacterium thermosulfurigenes and in Archaea Methanococcus jannaschii and Methanococcus voltae.

1. Use of substances which bind to EF-Tu to inhibit the formation of a cytoskeleton in bacterial cells.

2. Use as claimed in claim 1, characterized in that the substances bind to EF-Tu in the region of domain 2 (amino acids 205-298) or/and of domain 3 (amino acids 299 to 394).

3. Use as claimed in claim 1, characterized in that the substances bind to EF-Tu in the region of amino acids 218 to 224 of domain 2.

4. Use as claimed in claim 1, characterized in that the substances bind to EF-Tu in the region of amino acids 317 to 328 or/and 343 to 354 of domain 3.

5. Use as claimed in one of the claim 1, characterized in that the substances contain partial sections of the amino acid sequences of domains 2 or/and 3 having a length of 4 to 20 amino acids.

6. Use as claimed in claim 5, characterized in that the partial sections have a length of 5 to 15 amino acids, in particular 6 to 12 amino acids.

7. Use as claimed in claim 1, characterized in that the substances are selected from linear or cyclic peptide compounds or peptidomimetics.

8. Use as claimed in claim 7, characterized in that the peptidic compounds or peptidomimetics contain bound hydrophobic groups, very bulky groups or/and groups which protect against degradation.

9. Use as claimed in claim 1 to produce an antibacterial agent.

10. Use as claimed in claim 9, characterized in that the antibacterial agent is formulated as a pharmaceutical composition optionally together with common pharmaceutical vehicles, diluents or/and auxiliary substances.

11. Use as claimed in claim 9 to produce an agent against Gram-positive or Gram-negative bacteria.

12. Use as claimed in claim 9 to produce an agent against mycoplasma.

13. Antibacterial agent containing partial sections of amino acid sequences of domains 2 and/or 3 of the bacterial protein EF-Tu having a length of 4 to 20 amino acids.

14. Antibacterial agent as claimed in claim 13 containing partial sections having a length of 5 to 15 amino acids.

15. Antibacterial agent as claimed in claim 13 containing partial sections having a length of 6 to 12 amino acids.

16. Antibacterial agent as claimed in claim 13, characterized in that it contains hydrophobic groups bound to the partial sections, very bulky groups and/or groups which protect against degradation.

17. Antibacterial agent as claimed in claim 13, formulated as a pharmaceutical composition optionally together with common pharmaceutical vehicles, diluents or/and auxiliary substances.

18. Method for identifying new antibacterial substances comprising:
(a) contacting a substance to be tested with bacterial EF-Tu or with a partial fragment thereof capable of polymerization and (b) determining whether the substance can inhibit the formation of EF-Tu polymers.

19. Method as claimed in claim 18, characterized in that it is carried out as an in vitro test.

20. Method as claimed in claim 18, characterized in that it is carried out as an in vivo test.

21. Method as claimed in claim 18, characterized in that labeled antibodies or/and labeled EF-Tu proteins or polymerizable partial fragments thereof are used to determine the formation of EF-Tu polymers.

22. Method as claimed in claim 18, characterized in that a substance which inhibits the formation of EF-Tu polymers or a substance derived therefrom is formulated as a pharmaceutical composition optionally together with common pharmaceutical vehicles, diluents or/and auxiliary substances.
